(19) United States
(12) Patent Application Publication
(10) Pub. No.: US 2010/0256700 A1
(43) Pub. Date: Oct. 7, 2010

(54) METHOD AND APPARATUS FOR ORGAN SPECIFIC INFLAMMATION THERAPY

(76) Inventors: Allan C. Shuros, St. Paul, MN (US); Craig Stolen, New Brighton, MN (US); Shantha Arcot-Krishnamurthy, Vadnais Heights, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/755,041

(22) Filed: Apr. 6, 2010

Related U.S. Application Data
(60) Provisional application No. 61/167,280, filed on Apr. 7, 2009.

(51) Int. Cl. A61N 1/365; A61N 1/39

(52) U.S. Cl. 607/6; 607/17

(57) ABSTRACT

A method and device are described for delivering cardiac therapy in which an implantable device for delivering such cardiac therapy is additionally configured to detect the presence of inflammation. Upon detection of inflammation, the device may be configured to modify its delivery of therapy in various ways and/or to communicate the information to an external agent for other types of interventions.

### CROSS-REFERENCE TO RELATED APPLICATION

This application claims the benefit of U.S. Provisional Application No. 61/167,280, filed on Apr. 7, 2009, under 35 U.S.C. §119(e), which is hereby incorporated by reference in its entirety.

This application is related to U.S. Provisional Patent Application Ser. No. 61/099,251 filed on Sep. 23, 2008, hereby incorporated by reference.

### FIELD OF THE INVENTION

This invention pertains to apparatus and methods for the treatment and detection of disease, and in particular heart disease and to devices providing electrostimulation to the heart such as cardiac pacemakers.

### BACKGROUND

Heart failure (HF) is a debilitating disease that refers to a clinical syndrome in which an abnormality of cardiac function causes a below normal cardiac output that can fall below a level adequate to meet the metabolic demand of peripheral tissues. Heart failure can be due to a variety of etiologies with ischemic heart disease being the most common. Heart failure is usually treated with a drug regimen designed to augment cardiac function and/or relieve congestive symptoms.

Electrostimulation of the ventricles can also be useful in treating heart failure. It has been shown that some heart failure patients suffer from intraventricular and/or interventricular conduction defects (e.g., bundle branch blocks) such that their cardiac outputs can be increased by improving the synchronization of ventricular contractions with electrical stimulation. In order to treat these problems, implantable cardiac devices have been developed that provide appropriately timed electrical stimulation to one or more heart chambers in an attempt to improve the coordination of atrial and/or ventricular contractions, termed cardiac resynchronization therapy (CRT). Ventricular resynchronization is useful in treating heart failure because, although not directly inotropic, resynchronization can result in a more coordinated contraction of the ventricles with improved pumping efficiency and increased cardiac output. Currently, a most common form of CRT applies stimulation pulses to both ventricles, either simultaneously or separated by a specified biventricular offset interval, and after a specified atrio-ventricular delay interval with respect to the detection of an intrinsic atrial contraction or delivery of an atrial pace. Other types of electrostimulation may be useful for improving cardiac performance in HF patients by enhancing myocardial contractility.

Heart failure is also a disorder characterized in part by immune activation and inflammation. Thus, patients with HF have elevated levels of a number of inflammatory cytokines, both in the circulation and in the failing heart itself. Coronary artery disease (atherosclerotic plaque), which is often present in HF patients, is also associated with inflammation. Such inflammation has an effect on how various cardiac therapies delivered by an implantable device perform and may indicate the need to modify the delivery of such therapies.

### BRIEF DESCRIPTION OF THE DRAWINGS

FIG. 1 illustrates the physical configuration of an exemplary pacing device.

FIG. 2 shows the components of an exemplary device.

FIG. 3 is a block diagram of the electronic circuitry of an exemplary device.

FIG. 4 illustrates an exemplary treatment algorithm incorporating sensing of inflammation.

### DETAILED DESCRIPTION

Described herein is a method and device for delivering cardiac therapy in which an implantable device for delivering such cardiac therapy is additionally configured to detect the presence of inflammation. Upon detection of inflammation, the device may be configured to modify its delivery of therapy in various ways and/or to communicate the information to an external agent for other types of interventions. (An implantable device may also be configured to deliver other types of organ-specific therapy as modulated by detection of inflammation.) Described below are different types of cardiac therapy that may be delivered by a cardiac device, techniques by which the device may detect inflammation, the manners in which the different types of cardiac therapy may be modified in response to detection of inflammation, and an exemplary hardware platform.

### Detection of Inflammation

Inflammation in a particular organ is typically causes increased extravascular fluid in the affected tissue, which fluid is drained by the lymphatic system. The lymphatic circulation begins with highly permeable lymph capillaries that drain the lymph to larger contractile lymphatics, which have valves as well as smooth muscle walls. The functional unit of a lymph vessel is known as a lymphangion, which is the segment between two valves. The lymphangion is contractile, depending upon the ratio of its length to radius and wall thickness. An increased lymphangion contraction rate indicates increased lymphatic flow, which thus indicates the presence of inflammation. An increase in size of the lymph node into which a lymph vessel drains may also be used to detect inflammation.

An implantable device as described herein incorporates an inflammation sensor that senses information relating to lymphangion contraction or the size dimension of a lymphangion or lymph node. In one embodiment, a cardiac device equipped with an inflammation sensor modulates the manner in which therapy is delivered in accordance with detection of inflammation. As described below, such therapy modulation may entail changing parameter values used to delivery therapy as well as the choice of specific therapies. In another embodiment, the inflammation sensor is incorporated in a cardiac rhythm management system with a processor designed to trend the lymphangion contractile information. Algorithms using sensor information may detect decompensation events and provide alerts via a patient management system in which the information is communicated to a patient management server or elsewhere via telemetry and network communications. Algorithms using sensor information may also track the inflammatory response to various kinds of cardiac therapy such as cardiac resynchronization therapy and suggest therapy optimization programming changes. To prevent aggravating secondary disease, certain preventative cardiac therapies (such as intermittent pacing as described below) may be suspended in the presence of inflammatory situations. Inflammation monitoring as described can also be used for providing alerts to vulnerable plaque or pocket infections, general inflammation monitoring, and in the monitoring of Crohn's disease and sub-clinical hepatitis.

The inflammation sensor as described herein uses sensed lymphangion contraction or the size dimension of a lymphangion or lymph node as a measure of up-stream inflammation (i.e., inflammation in the specific organ drained by a particular lymphangion). An increase in the rate and/or magnitude (i.e., strength) of lympangion contraction may be indicative of inflammation. Lymphangion contraction may be detected by, for example: 1) a change in size of the lymphangion or lymph node (increased diameter, strain), 2) a change in pressure (increased interstitial pressure), 3) a change in lymphangion wall thickness (optical), 4) video recordings of downward-going deflections, 5) a change in the lymphangion impedance, and 6) lymphangion myopotentials. The lymphatic sensor for detecting one or more of the above-listed variables may be one or more electrodes, an optical sensor, a pressure sensor, or an acoustic sensor. The sensor may be connected via an implantable lead to the housing and electronic circuitry of the implantable device or may be a satellite device that communicates wirelessly with the implantable device. In order to detect inflammation, the lymphatic sensor signal may be compared to a specified threshold. The amount of inflammation present may also be quantified from the lymphatic sensor signal according to a specified scale.

### Response to Inflammation Detection by Different Types of Cardiac Therapy

The most common type of electrostimulatory therapy delivered by implantable cardiac devices is pacing therapy delivered to selected chambers of the heart in order to treat disorders of cardiac rhythm. A pacemaker, for example, is a cardiac rhythm management device that paces the heart with timed pacing pulses. The most common condition for which pacemakers have been used is in the treatment of bradycardia, where the ventricular rate is too slow. Atrio-ventricular conduction defects (i.e., AV block) that are permanent or intermittent and sick sinus syndrome represent the most common causes of bradycardia for which permanent pacing may be indicated. If functioning properly, the pacemaker makes up for the heart's inability to pace itself at an appropriate rhythm in order to meet metabolic demand by enforcing a minimum heart rate and/or artificially restoring AV conduction. As noted above, pacing therapy may also be used in the treatment of cardiac conduction disorders in order to improve the coordination of cardiac contractions, termed cardiac resynchronization therapy. Other cardiac rhythm management devices are designed to detect atrial and/or ventricular tachyarrhythmias and deliver electrical stimulation in order to terminate the tachyarrhythmia in the form of a cardioversion/defibrillation shock or anti-tachycardia pacing. Certain combination devices may incorporate all of the above functionalities. Any device with a pacing functionality will be referred to herein simply as a pacemaker regardless of other functions it may be capable of performing.

Another form of electrostimulatory treatment that may be delivered by an implantable device is neural stimulation to a nerve innervating the heart such as the vagus nerve or a sympathetic nerve. For example, the vagus nerve provides parasympathetic stimulation to the heart which counteracts the effects of increased sympathetic activity in a manner which is beneficial when the blood supply to the heart is compromised. Stimulation of the vagus nerve at either a pre-ganglionic or post-ganglionic site produces dilation of the coronary arteries and a reduced workload on the heart via its effect on myocardial contractility.

It has also been demonstrated that electrostimulatory pulses may be delivered to the heart in a manner that can augment myocardial contractility, sometimes referred to as cardiac contractility modulation (CCM). Applying contractility augmenting stimulation to the ventricles can thus aid in the treatment of heart failure. Contractility augmenting stimulation can be delivered during the refractory period after an intrinsic contraction and hence is non-excitatory stimulation. Presumably, such stimulation increases myocardial contractility by increasing intracellular calcium concentration and/or eliciting release of neurotransmitters. Contractility augmenting stimulation can also be applied in an excitatory manner, however, referred to herein as high-output pacing (HOP). In one form of HOP, the stimulation is delivered in the same manner as conventional pacing using a bradycardia pacing mode using stimulation pulses with a higher stimulation energy. For example, a stimulation pulse for high-output pacing may be a biphasic (or multiphasic) waveform having a peak-to-peak voltage amplitude of + or −5-8 volts and a pulse duration of 20-70 milliseconds. In another form of HOP, similar stimulation pulses are delivered in the refractory period following a conventional ventricular pacing pulse. Further descriptions of devices that delivery HOP therapy and which may incorporate the subject matter of the present application are found in commonly assigned U.S. patent application Ser. Nos. 11/860,957 filed on Sep. 25, 2007 and 61/090,485 filed on Aug. 20, 2008, hereby incorporated by reference.

Another form of electrostimulatory therapy is pacing delivered in a manner that advantageously redistributes myocardial stress during systole for therapeutic purposes in the treatment of, for example, patients with ischemic heart disease, post-MI patients, and HF patients. Myocardial regions that contract earlier during systole experience less wall stress than later contracting regions. Pacing pulses may be delivered to a particular myocardial region to pre-excite that region relative to other regions during systole, with the latter being excited by intrinsic activation or a subsequent pacing pulse. (As the term is used herein, a pacing pulse is any type of electrical stimulation that excites the myocardium, whether or not used to enforce a particular rate.) As compared with an intrinsic contraction, the pre-excited region is mechanically unloaded or de-stressed, while the later excited regions are subjected to increased stress. Such pre-excitation pacing may be applied to deliberately de-stress a particular myocardial region that may be expected to undergo deleterious remodeling, such as the area around a myocardial infarct or a hypertrophying region. Pre-excitation pacing may also be applied to deliberately stress a region remote from the pre-excitation pacing site in order to exert a conditioning effect, similar to the beneficial effects of exercise. Whether for intentionally stressing or de-stressing a myocardial region, such cardioprotective pre-excitation pacing may be applied intermittently, either according to a defined schedule or upon detection of specified entry or exit conditions, and is referred to herein as intermittent pacing therapy or IPT. Further descriptions of devices that delivery IPT and which may incorporate the subject matter of the present application are found in commonly assigned U.S. patent application Ser. Nos. 11/689,032 filed on Mar. 21, 2007 and 11/687,957 filed on Mar. 19, 2007, hereby incorporated by reference.

As noted above, detection of inflammation may indicate a need for modifying the therapy delivered by a cardiac device. Since inflammation can affect the capture threshold of myocardial tissue, a device that delivers pacing stimulation (i.e., electrostimulation for causing myocardial contraction) may be configured to increase the pulse energy (e.g., by increasing pacing pulse amplitude or duration) upon detection of inflammation. It has also been shown that inflammation can affect how patients respond to CRT therapy. A CRT device may therefore be programmed to modify its therapy upon detection of inflammation by changing CRT parameters (e.g., atrio-ventricular delay or biventricular offset interval) or by initiating or ceasing particular therapy modes. Inflammation may also indicate an ischemic event for which neural or certain types of cardiac stimulation may be beneficially initiated. It has also been found that inflammation can increase the likelihood of sudden cardiac death in certain patients. A device configured to deliver anti-tachyarrhythmia therapy such as cardioversion/defibrillation shocks or anti-tachcardia pacing may therefore be programmed to increase the sensitivity and decrease the specificity for detection of a tachyarrhythmia by decreasing a tachyarrhythmia threshold upon detection of inflammation.

Described above are devices that modify their therapy based upon detection of inflammation. A device configured to deliver a variable amount of therapy may also be configured to modify that amount based upon a quantized measure of the amount of inflammation present as derived from the lymphatic sensor signal. For example, a device that delivers a particular mode of therapy according to a timed duty cycle (e.g., intermittent pacing therapy, CRT, neural stimulation, HOP) may be programmed to increase or decrease the duty cycle in accordance with the amount of inflammation detected. The device could also be programmed to use the amount of inflammation detected as a feedback variable for adjusting therapy parameters. For example, the device could deliver CRT, IPT, HOP, and/or neural stimulation with a specified set of therapy parameters (i.e., parameters relating to pulse energy, pulse timing, and/or therapy duty cycles) as long as the inflammation level remains constant or is decreasing but to adjust the parameters if inflammation is increasing.

### Exemplary Cardiac Device

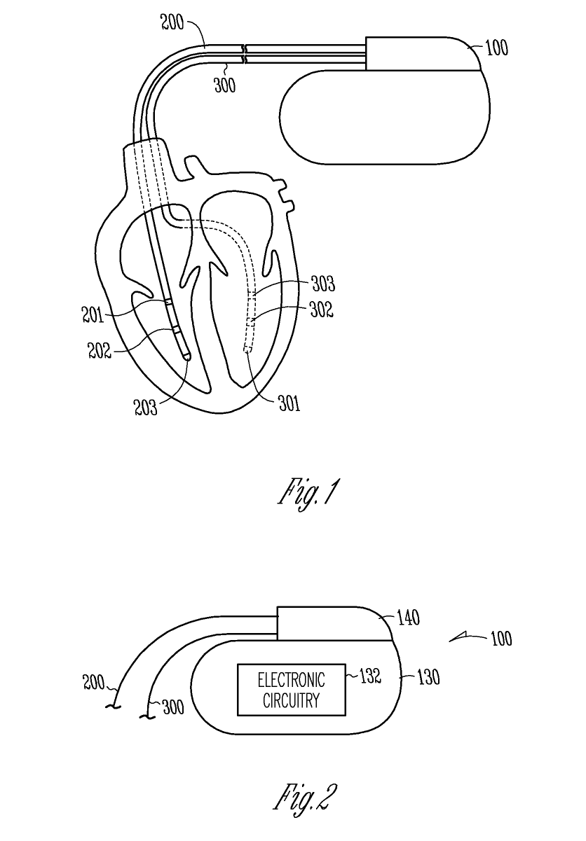

FIG. 1 shows an implantable cardiac pacing device 100 for delivering pacing therapy of any of the types described above. Implantable pacing devices are typically placed subcutaneously or submuscularly in a patient's chest with leads threaded intravenously into the heart to connect the device to electrodes disposed within a heart chamber that are used for sensing and/or pacing of the chamber. Electrodes may also be positioned on the epicardium by various means. A programmable electronic controller causes the pacing pulses to be output in response to lapsed time intervals and/or sensed electrical activity (i.e., intrinsic heart beats not as a result of a pacing pulse). The device senses intrinsic cardiac electrical activity through one or more sensing channels, each of which incorporates one or more of the electrodes. In order to excite myocardial tissue in the absence of an intrinsic beat, pacing pulses with energy above a certain threshold are delivered to one or more pacing sites through one or more pacing channels, each of which incorporates one or more of the electrodes. FIG. 1 shows the exemplary device having two leads 200 and 300, each of which is a multi-polar (i.e., multi-electrode) lead having electrodes 201-203 and 301-303, respectively. The electrodes 201-203 are disposed in the right ventricle in order to stimulate or sense right ventricular or septal regions, while the electrodes 301-303 are disposed in the coronary sinus in order to stimulate or sense regions of the left ventricle. Other embodiments may use any number of electrodes in the form of unipolar and/or multi-polar leads in order to sense or stimulate different myocardial sites. Once the device and leads are implanted, the stimulation and/or sensing channels of the device may be configured with selected ones of the multiple electrodes in order to selectively stimulate or sense a particular myocardial site(s). The stimulation channels may be used to deliver conventional bradycardia pacing, CRT, HOP therapy, intermittent stress augmenting pacing therapy, and/or neural stimulation.

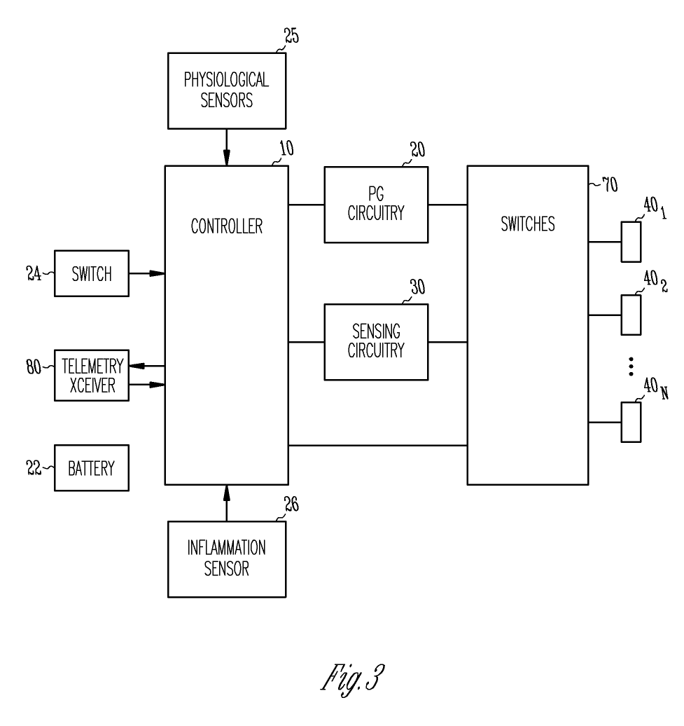

FIG. 2 shows the components of the implantable device 100 in more detail. The implantable device 100 includes a hermetically sealed housing 130 that is placed subcutaneously or submuscularly in a patient's chest. The housing 130 may be formed from a conductive metal, such as titanium, and may serve as an electrode for delivering electrical stimulation or sensing in a unipolar configuration. A header 140, which may be formed of an insulating material, is mounted on the housing 130 for receiving leads 200 and 300 which may be then electrically connected to pulse generation circuitry and/or sensing circuitry. Contained within the housing 130 is the electronic circuitry 132 for providing the functionality to the device as described herein which may include a power supply, sensing circuitry, pulse generation circuitry, a programmable electronic controller for controlling the operation of the device, and a telemetry transceiver capable of communicating with an external programmer or a remote monitoring device.

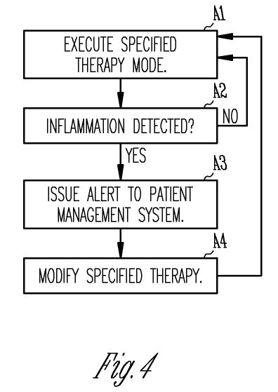

FIG. 3 shows a system diagram of the electronic circuitry 132. A battery 22 supplies power to the circuitry. The controller 10 controls the overall operation of the device in accordance with programmed instructions and/or circuit configurations, including decisions as to the time and manner of stimulation pulses through the stimulation channels. The controller may be implemented as a microprocessor-based controller and include a microprocessor and memory for data and program storage, implemented with dedicated hardware components such as ASICs (e.g., finite state machines), or implemented as a combination thereof. The controller also includes timing circuitry such as external clocks for implementing timers used to measure lapsed intervals and schedule events. As the term is used herein, the programming of the controller refers to either code executed by a microprocessor or to specific configurations of hardware components for performing particular functions. Interfaced to the controller are sensing circuitry 30 and pulse generation circuitry 20 by which the controller interprets sensing signals and controls the delivery of paces and/or other stimulation pulses in accordance with a pacing mode. The controller is capable of operating the device in a number of programmed pacing modes which define how pulses are output in response to sensed events and expiration of time intervals. The controller also implements timers derived from external clock signals in order to keep track of time and implement real-time operations such as scheduled entry into a particular type of therapy mode.

The sensing circuitry 30 receives atrial and/or ventricular electrogram signals from sensing electrodes and includes sensing amplifiers, analog-to-digital converters for digitizing sensing signal inputs from the sensing amplifiers, and registers that can be written to for adjusting the gain and threshold values of the sensing amplifiers. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing, and the intrinsic atrial and/or ventricular rates can be detected by measuring the time intervals between atrial and ventricular senses, respectively. The pulse generation circuitry 20 delivers conventional pacing, HOP, or other stimulation pulses to electrodes disposed in the heart or elsewhere and includes capacitive discharge or current source pulse generators, registers for controlling the pulse generators, and registers for adjusting parameters such as pulse energy (e.g., pulse amplitude and width). The pulse generation circuitry may also include a shocking pulse generator for delivering a defibrillation/cardioversion shock via a shock electrode upon detection of a tachyarrhythmia.

A telemetry transceiver 80 is interfaced to the controller which enables the controller to communicate with an external device such as an external programmer and/or a remote monitoring unit. An external programmer is a computerized device with an associated display and input means that can interrogate the pacemaker and receive stored data as well as directly adjust the operating parameters of the pacemaker. The external device may also be a remote monitoring unit that may be interfaced to a patient management network enabling the implantable device to transmit data and alarm messages to clinical personnel over the network as well as be programmed remotely. The network connection between the external device and the patient management network may be implemented by, for example, an internet connection, over a phone line, or via a cellular wireless link. A switch 24 is also shown as interfaced to the controller in this embodiment to allow the patient to signal certain conditions or events to the implantable device. In different embodiments, the switch 24 may be actuated magnetically, tactilely, or via telemetry such as by a hand-held communicator. The controller may be programmed to use actuation of the switch 24 to as an entry and/or exit condition for entering a particular therapy mode.

A stimulation channel is made up of a pulse generator connected to an electrode, while a sensing channel is made up of a sense amplifier connected to an electrode. Shown in the figure are electrodes 40₁ through 40_{N} where N is some integer. The electrodes may be on the same or different leads and are electrically connected to a MOS switch matrix 70. The switch matrix 70 is controlled by the controller and is used to switch selected electrodes to the input of a sense amplifier or to the output of a pulse generator in order to configure a sensing or pacing channel, respectively. The device may be equipped with any number of pulse generators, amplifiers, and electrodes that may be combined arbitrarily to form sensing or pacing channels. The device is therefore capable of delivering single-site or multiple site ventricular pacing and/or other type of stimulation. The switch matrix 70 also allows selected ones of the available implanted electrodes to be incorporated into sensing and/or pacing channels in either unipolar or bipolar configurations. A bipolar sensing or pacing configuration refers to the sensing of a potential or output of a pacing pulse between two closely spaced electrodes, where the two electrodes are usually on the same lead (e.g., a ring and tip electrode of a bipolar lead or two selected electrodes of a multi-polar lead). A unipolar sensing or pacing configuration is where the potential sensed or the pacing pulse output by an electrode is referenced to the conductive device housing or another distant electrode.

The device may also include one or more physiological sensing modalities 25 for use in controlling, for example, the pacing rate, optimization of stimulation parameters, and/or the initiation/cessation of a particular therapy mode. One such sensing modality is an accelerometer that enables the controller to detect changes in the patient's physical activity, detect patient posture (i.e., using a multi-axis accelerometer), and/or detect heart sounds. A dedicated acoustic sensor that may be of various types may also be used to detect heart sounds. An impedance sensor may be configured with electrodes for measuring minute ventilation for use in rate adaptive pacing and/or for measuring cardiac stroke volume or cardiac output. The device may also include a pressure sensor that may be used, for example, to measure pressure in the pulmonary artery or elsewhere.

The device also includes an inflammation sensor 26 for detecting the presence of inflammation for detecting inflammation in a specific organ such as the heart. The sensor 26 may be connected to the controller 10 via an implantable lead or may be incorporated into an implantable satellite device that communicates wirelessly with the controller 10. The inflammation sensor 26 may include one or more electrodes for sensing myopotentials or measuring impedance, an optical or acoustic sensor for detecting the size or change in size of a lymphangion or lymph node, or a pressure sensor for sensing fluid pressure within a lymphangion or lymph node.

### Exemplary Embodiments

In an exemplary embodiment, an implantable device includes an inflammation sensor for detecting a physical characteristic of a lymphangion or lymph node interfaced to a controller configured to detect the presence of inflammation from signals generated by the inflammation sensor. The device also includes sensing and stimulation channels for sensing cardiac activity and delivering electrical stimulation to one or more myocardial or neural sites, where the controller is configured to deliver electrical stimulation via the one or more stimulation channels in accordance with one or more specified therapy modes. The controller is additionally programmed to modify the delivery of electrical stimulation when inflammation is detected.

In various particular embodiments that may be combined, the inflammation sensor may be configured to sense a size dimension of a lymphangion or lymph node, where the controller is programmed to detect inflammation if the size dimension is above a specified threshold or may be to sense contractions of a lymphangion, where the controller is programmed to detect inflammation if the lymphangion contraction rate and/or strength is above a specified threshold. The contraction of a lymphangion may be sensed by measuring a change in size of the lymphangion such as increased diameter, a change in mechanical strain, a change in pressure such as due to increased interstitial pressure, a change in lymphangion wall thickness measured optical, a change detected via video recordings of downward-going deflections, a change in the lymphangion impedance, or detection of lymphangion myopotentials. The inflammation sensor may be, for example, one or more electrodes for sensing myopotentials, an electrical impedance sensor, an acoustic transducer, or an optical sensor.

The implantable device may further include a telemetry unit for receiving inputs from an external device, where the controller is further programmed to detect the presence of inflammation based upon signals from the inflammation sensor and inputs received from the external device. The telemetry unit may also be used to issue alerts to an external device such as an external patent management system upon detection of inflammation.

The therapy delivered by the device may be bradycardia pacing, cardiac resynchronization pacing, intermittent pacing therapy, contractility augmenting pacing, and neural stimulation. The controller may be programmed to increase the amplitude or duration of stimulation pulses used in any of these therapy modes upon detection of increased inflammation. If the specified therapy mode is contractility enhancement stimulation (e.g., high-output pacing, anodal pacing, or refractory period stimulation), the controller may be programmed to initiate or otherwise increase the extent of such contractility enhancement stimulation upon detection of increased inflammation. If the specified therapy mode is intermittent stress augmentation pacing, the controller may be programmed to discontinue or otherwise reduce the extent of such intermittent stress augmentation pacing upon detection of increased inflammation. If the specified therapy mode is cardiac resynchronization pacing, the controller may be programmed to re-optimize pacing parameters selected from a group that includes an atrio-ventricular delay interval and a biventricular offset interval upon detection of inflammation. If the specified therapy mode is delivery of anti-tachyarrhythmia therapy selected from a group that includes delivery of cardioversion/defibrillation shocks and delivery of anti-tachycardia pacing in response to detection of a tachyarrhythmia, the controller may be programmed to decrease a tachyarrhythmia threshold for detecting a tachyarrhythmia upon detection of increased inflammation and/or increase a tachyarrhythmia threshold upon detection of decreased inflammation. In any of the specified therapy modes discussed above, the controller could also be programmed such that the therapy mode adjustments made in response to increased inflammation are temporary and/or programmed to adjust pacing or other therapy parameters such as duty cycles in an opposite manner if a decreased level of inflammation is detected.

FIG. 4 illustrates an exemplary treatment algorithm incorporating sensing of inflammation. At step A4, the device operates to deliver therapy in a specified therapy mode while concurrently monitoring for the presence of inflammation at step A2. If inflammation is detected, the device issues an alert via telemetry to a patient management system at step A3. The specified therapy is also modified in any of the manners described above at step A4 before returning to step A1.

The invention has been described in conjunction with the foregoing specific embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. An implantable device, comprising:
sensing and stimulation channels for sensing cardiac activity and delivering electrical stimulation to one or more myocardial or neural sites;
a controller configured to deliver electrical stimulation via one or more pacing channels in accordance with one or more specified therapy modes;
an inflammation sensor for detecting a physical characteristic of a lymphangion or lymph node;
wherein the controller is configured to detect the presence of inflammation from signals generated by the inflammation sensor; and,
wherein the controller is programmed to modify the delivery of electrical stimulation when inflammation is detected.

2. The implantable device of claim 1 wherein the inflammation sensor is configured to sense a size dimension of a lymphangion or lymph node, and wherein the controller is programmed to detect inflammation if the size dimension is above a specified threshold.

3. The implantable device of claim 1 wherein the inflammation sensor is configured to sense contractions of a lymphangion.

4. The implantable device of claim 3 wherein the controller is programmed to detect inflammation if the lymphangion contraction rate is above a specified threshold.

5. The implantable device of claim 3 wherein the controller is programmed to detect inflammation if the lymphangion contraction strength is above a specified threshold.

6. The implantable device of claim 3 wherein the inflammation sensor is configured to sense the contraction of a lymphangion by measuring a change in size of the lymphangion such as increased diameter, a change in mechanical strain, a change in pressure such as due to increased interstitial pressure, a change in lymphangion wall thickness measured optical, a change detected via video recordings of downward-going deflections, a change in the lymphangion impedance, or detected lymphangion action potentials.

7. The implantable device of claim 1 wherein the inflammation sensor is of a type selected from a group that includes one or more electrodes for sensing myopotentials, an electrical impedance sensor, an acoustic transducer, and an optical sensor.

8. The implantable device of claim 1 further comprising a telemetry unit for receiving inputs from an external device and wherein the controller is further programmed to detect the presence of inflammation based upon signals from the inflammation sensor and inputs received from the external device.

9. The implantable device of claim 1 wherein the one or more specified therapy modes are selected from a group that includes bradycardia pacing, cardiac resynchronization pacing, intermittent pacing therapy, contractility augmenting pacing, and neural stimulation.

10. The implantable device of claim 1 wherein the controller is programmed to increase the amplitude or duration of stimulation pulses upon detection of inflammation.

11. The implantable device of claim 1 wherein the specified therapy mode is contractility enhancement stimulation selected from a group that includes high-output pacing, anodal pacing, and refractory period stimulation and wherein the controller is programmed to initiate or otherwise increase the extent of contractility enhancement stimulation upon detection of inflammation.

12. The implantable device of claim 1 wherein the specified therapy mode is intermittent stress augmentation pacing and wherein the controller is programmed to discontinue or otherwise reduce the extent of intermittent stress augmentation pacing upon detection of inflammation.

13. The implantable device of claim 1 wherein the specified therapy mode is cardiac resynchronization pacing and wherein the controller is programmed to re-optimize pacing parameters selected from a group that includes an atrio-ventricular delay interval and a biventricular offset interval upon detection of inflammation.

14. The implantable device of claim 1 wherein the specified therapy mode is delivery of anti-tachyarrhythmia therapy selected from a group that includes delivery of cardioversion/defibrillation shocks and delivery of anti-tachycardia pacing in response to detection of a tachyarrhythmia and wherein the controller is programmed to decrease a tachyarrhythmia threshold for detecting a tachyarrhythmia upon detection of increased inflammation.

15. The implantable device of claim 1 wherein the specified therapy mode is delivery of anti-tachyarrhythmia therapy selected from a group that includes delivery of cardioversion/defibrillation shocks and delivery of anti-tachycardia pacing in response to detection of a tachyarrhythmia and wherein the controller is programmed to increase a tachyarrhythmia threshold for detecting a tachyarrhythmia upon detection of decreased inflammation.

16. The implantable device of claim 1 wherein the specified therapy mode is selected from a group that includes cardiac resynchronization pacing, intermittent pacing therapy, contractility augmenting pacing, and neural stimulation using a specified set of therapy parameters, and wherein the controller is programmed to adjust the therapy parameters if an increased or decreased amount of inflammation is detected.

17. A method for operating an implantable device, comprising:
delivering electrical stimulation via one or more stimulation channels to one or more myocardial or neural sites in accordance with one or more specified therapy modes;
employing an inflammation sensor to detect a physical characteristic of a lymphangion or lymph node;
detecting the presence of inflammation from signals generated by the inflammation sensor; and,
modifying the delivery of electrical stimulation when inflammation is detected.

18. The method of claim 17 further comprising employing the inflammation sensor to sense a size dimension of a lymphangion or lymph node and detecting inflammation if the size dimension is above a specified threshold.

19. The method of claim 17 further comprising employing the inflammation sensor to detect inflammation if the lymphangion contraction rate is above a specified threshold.

20. The method of claim 17 further comprising employing the inflammation sensor to detect inflammation if the lymphangion contraction strength is above a specified threshold.
